(19) United States
(12) Patent Application Publication
(10) Pub. No.: US 2026/0166197 A1
(43) Pub. Date: Jun. 18, 2026

(54) SILVER HYDROGEL BIOCERAMIC COMPOSITIONS FOR DENTAL AND MEDICAL APPLICATIONS

(71) Applicant: Innovative BioCeramix Inc., Burnaby (CA)

(72) Inventor: Donghui LU, Vancouver (CA)

(21) Appl. No.: 19/422,339

(22) Filed: Dec. 16, 2025

Related U.S. Application Data
(60) Provisional application No. 63/833,573, filed on Dec. 17, 2024.

(51) Int. Cl. A61L 24/00; B82Y 5/00

(52) U.S. Cl. CPC A61L 24/0068; A61L 24/0015; A61L 24/0031; A61L 2400/12; B82Y 5/00

(57) ABSTRACT

The disclosure provides silver hydrogel-bioceramic premixed pastes, processes for manufacturing said premixed pastes, and uses of said premixed pastes in dental and/or medical applications. The premixed paste comprises at least one silver nanoparticle and/or at least one silver ion, at least one hydrogel former, at least one hydratable bioceramic material, and at least one non-aqueous liquid carrier that is hydrophilic and able to undergo an exchange with a water-based liquid when the premixed paste is exposed to an environment where water-based liquids are present.

### FIELD

The present disclosure relates generally to bioceramic compositions and their use in dental and/or medical applications.

### BACKGROUND

The following paragraphs are not an admission that anything discussed in them is prior art or part of the knowledge of persons skilled in the art.

Inorganic antimicrobial agents such as silver have been widely used in medical and dental materials due to their broad-spectrum antibacterial activity and chemical stability. Silver nanoparticles and silver ions exhibit antimicrobial properties and biocompatibility, making them desirable additives in bioceramic materials used for tissue repair and root canal sealing.

Bioceramic materials, including calcium silicates, calcium phosphates, and bioactive glasses, have biocompatibility, bioactivity, and osteoconductivity properties. These materials are capable of forming hydroxyapatite on their surface when exposed to body fluids, thus promoting tissue regeneration.

### INTRODUCTION

The following introduction is intended to introduce the reader to this specification but not to define any invention. One or more inventions may reside in a combination or sub-combination of the composition elements or method steps described below or in other parts of this document. The inventors do not waive or disclaim their rights to any invention or inventions disclosed in this specification merely by not describing such other invention or inventions in the claims.

Premixed bioceramic pastes, such as those disclosed by Innovative BioCeramix Inc. (for example, U.S. Pat. No. 8,475,811 B2 and EP 2142225 B1, inventors Quanzu Yang and Donghui Lu; herein incorporated by reference), introduced the concept of a non-aqueous, premixed hydraulic cement composition that remains stable in storage and hardens upon contact with moisture. These compositions have demonstrated excellent sealing and bioactive properties for endodontic applications. These pastes also incorporated antimicrobial agents including silver or silver oxide. However, these pastes are not taught as incorporating silver nanoparticles into a hydrogel structure for controlled release. Furthermore, if silver nanoparticles are directly incorporated into cement paste without encapsulation or trapping, e.g., via a hydrogel structure, the silver nanoparticles are easily leached out from the cement, and there is no controlled release of silver nanoparticles. The antimicrobial properties of the silver nanoparticles are thus short-lived as they have a short half-life. Overall, these previously disclosed pastes do not incorporate silver nanoparticles and/or silver ions in hydrogels into such premixed bioceramic systems to provide controlled and sustained antimicrobial performance.

Mestieri et al. disclosed research related to biocompatibility and bioactivity of calcium silicate-based endodontic sealers in human dental pulp cells (J Appl Oral Sci. 2015 September-October; 23 (5): 467-471). The research results indicated that calcium silicate materials are biocompatible and bioactive. Huang et al. disclosed research related to substitutions of strontium in bioactive calcium silicate bone cements to stimulate osteogenic differentiation in human mesenchymal stem cells (J Mater Sci Mater Med. 2019 Jun. 4; 30 (6): 68.). However, neither of these references disclose, teach, or suggest the use of silver nanoparticles and/or silver ions for dental or medical applications.

Torabinejad et al. (U.S. Pat. No. 5,415,547) disclosed a tooth filling material and a method of use. The method related to the filling and sealing of tooth cavities using a cement composition which exhibited several advantages over existing orthograde and retrograde filling materials, including the ability to set in an aqueous environment. In a preferred embodiment, the cement composition comprised Portland cement, or variations in the composition of such cement, which exhibited favorable physical attributes sufficient to form an effective seal against reentrance of infectious organisms. However, Torabinejad et al. did not disclose, teach, or suggest the incorporation of silver nanoparticles and/or silver ions into the cement composition for dental or medical applications.

Chow et al. (U.S. Pat. No. 9,101,436) disclosed endodontic filling materials and methods related thereto. One of the disclosed methods for filling a dental root canal included providing a hydrosetting filling material and inserting the hydrosetting filling material into the dental root canal so that the material sets in the root canal to form a biocompatible filling. The hydrosetting filling material comprised a hydrogel former and a filler. The hydrogel former was at least one of a reactive organic hydrogel former, an inorganic hydrogel former, or a non-reactive organic hydrogel former. Plural filling material precursor compositions that collectively contained hydrogel formers and fillers could be provided. Chow et al. taught the use of calcium silicates and sodium silicates as inorganic hydrogel formers. However, Chow et al. did not disclose, teach, or suggest any method or process for using, or provide a sample that used, calcium silicate and calcium phosphate as inorganic formers to make premixed paste without an organic hydrogel former because of inorganic hydrogel having a longer setting time. Chow et al. did not disclose, teach, or suggest the use of a silver nanoparticle and/or silver hydrogel in the endodontic filling materials.

Jia et al. (U.S. Pat. No. 7,303,817) disclosed a dental filling material comprising an inner core and an outer layer of material disposed and surrounding the inner core, wherein both the inner core and outer layer of material each contained a thermoplastic polymer. The thermoplastic polymer could be biodegradable, and a bioactive substance could also be included in the filling material. The thermoplastic polymer acted as a matrix for the bioactive substance. The dental filling material could include other polymeric resins, fillers, plasticizers and other additives typically used in dental materials. The dental filling material was used for the filling of root canals. However, Jia et al. did not disclose, teach, or suggest the use of silver nanoparticles and/or silver ions for dental or medical applications.

Wagh et al. (U.S. Pat. No. 7,083,672) disclosed a phosphosilicate slurry composition for use in dentistry and related bone cements, and a method of producing said composition. The composition was produced by combining a mixture of a substantially dry powder component with a liquid component. The substantially dry powder component comprised a sparsely soluble oxide powder, an alkali metal phosphate powder, and a sparsely soluble silicate powder, with the balance of the substantially dry powder component comprising at least one powder selected from the group consisting of bioactive powders, biocompatible powders, fluorescent powders, fluoride releasing powders, and radiopaque powders. The liquid component comprised a pH modifying agent, e.g., a monovalent alkali metal phosphate in aqueous solution, the balance of the liquid component being water. The use of calcined magnesium oxide as the oxide powder and hydroxyapatite as the bioactive powder produced a self-setting ceramic that was particularly suited for use in dental and orthopedic applications. Calcium silicate was disclosed as a sparsely soluble (in water) powder. However, Wagh et al. did not disclose, teach, or suggest the use of silver nanoparticles and/or silver ions for dental or medical applications.

Zhang (US20150265509A1) disclosed a method of making silver nanoparticles and their applications. Zhang disclosed a micro particle with a diameter of 10-100 microns, wherein the micro particle was coated with silver nanoparticles, wherein the nanoparticles were coated with a polysaccharide, and wherein the polysaccharide coating was digestible by bacteria. However, Zhang did not disclose, teach, or suggest that the coated microparticles were suitable for dental filling and repair applications, or that the coated microparticles may be encapsulated or trapped in a hydrogel structure.

Overall, existing silver-containing bioceramics generally rely on direct doping or surface coating of silver compounds onto the ceramic phase, which can result in rapid silver release, particle aggregation, and/or reduced biocompatibility. The color of silver nanoparticles is gray or black. When silver nanoparticles are directly incorporated into paste materials, as disclosed in one or more existing methods, they impart a gray or black coloration to the paste, which may also cause tooth discoloration. These limitations highlight the need for an improved system capable of stabilizing silver nanoparticles or silver ions, preventing agglomeration, ensuring sustained antimicrobial activity without impairing the setting or bioactivity of the bioceramic phase, or any combination thereof.

As previously stated, silver nanoparticles and silver ions are desirable additives in bioceramic materials used for tissue repair and root canal sealing due to their effective antimicrobial properties and biocompatibility. However, the incorporation of silver nanoparticles and/or silver ions into ceramics using one or more existing methods may result in silver leaching out of the composition at uncontrolled rates; and the direct incorporation of silver nanoparticles and/or silver ions into aqueous environments often results in their uncontrolled release, particle agglomeration, and/or precipitation, which can compromise stability and reduce long-term efficacy.

The present disclosure provides a premixed, hydrogel-bioceramic composition that integrates the: (1) antimicrobial, anti-inflammatory, and stable release characteristics of silver hydrogels; and (2) bioactive and osteoconductive characteristics of hydratable bioceramics. Such a composition provides enhanced handling, long-term storage stability, and/or multifunctional performance suitable for dental and/or medical applications. The hydrogel-bioceramic composition may be in the form of a paste before hydration.

The present disclosure provides a silver hydrogel-bioceramic composition, its method of manufacture, and its use in dental and/or medical applications. The silver hydrogel-bioceramic composition is an advanced material that combines the properties of silver particles, hydrogels, and bioceramic components to create a multifunctional system with applications in biomedicine, tissue engineering, dental applications, and/or wound care. The composition comprises at least one silver nanoparticle and/or at least one silver ion, at least one hydrogel former, at least one hydratable bioceramic material, and at least one non-aqueous liquid carrier, to form a multifunctional premixed paste. Upon exposure to an aqueous or biological environment, the non-aqueous liquid carrier is replaced by water or moisture, initiating hydrogel formation and bioceramic hydration. The formed hydrogel (also referred to as a hydrogel matrix, hydrogel network, or hydrogel structure) acts as a coupling agent between the silver nanoparticles and/or silver ions and the bioceramic material. The formed hydrogel may also act to encapsulate or trap and stabilize the silver nanoparticles and/or silver ions, enabling their controlled release. In some examples, the hydrogel is formed and encapsulates or traps and stabilizes the silver nanoparticles and/or silver ions before the paste is exposed to a biological environment. The resulting hydrated composition exhibits biocompatibility, bioactivity, antimicrobial, anti-inflammatory, and/or osteoconductive properties, making it suitable for root canal filling, bone repair, and/or other dental and orthopedic applications. Also, the encapsulation or entrapment of the silver nanoparticles and/or silver ions within the hydrogel can increase the masking of the gray or black color of the silver nanoparticles and/or silver ions.

Other aspects and features of the present disclosure will become apparent to those ordinarily skilled in the art upon review of the following description and specific examples.

### DETAILED DESCRIPTION

Generally, the present disclosure provides a silver hydrogel-bioceramic composition for use in medical and/or dental applications, comprising: at least one silver nanoparticle and/or at least one silver ion; at least one hydrogel former; at least one hydratable bioceramic material; and at least one non-aqueous liquid carrier that is hydrophilic and able to undergo an exchange with a water-based fluid when the premixed paste is exposed to an environment where water-based fluids are present, for example, a physiological environment.

The present disclosure also provides a method of forming a premixed paste for use in medical and/or dental applications, the method comprising the steps of: mixing: at least one silver nanoparticle and/or at least one silver ion; at least one hydrogel former; at least one hydratable bioceramic material; and at least one non-aqueous liquid carrier that is hydrophilic and able to undergo an exchange with a water-based fluid when the premixed paste is exposed to an environment where water-based fluid is present.

The present disclosure also provides a method for forming a cementitious mass in medical and/or dental applications, said method comprising the steps of: providing a premixed paste, said premixed paste comprising: at least one silver nanoparticle and/or at least one silver ion; at least one hydrogel former; at least one hydratable bioceramic material; and at least one non-aqueous liquid carrier that is hydrophilic and able to undergo an exchange with a water-based liquid when the premixed paste is exposed to an environment where water-based fluid is present; and placing said premixed paste in a physiological environment so that said at least one non-aqueous liquid carrier undergoes exchange with an aqueous physiological fluid so that said paste hydrates in said physiological environment.

When the herein disclosed premixed paste is placed in a biological environment, the non-aqueous liquid carrier exchanges by diffusion with water and/or moisture from a biological environment, and the hydrogel former absorbs the water and/or moisture to form a hydrogel. The silver nanoparticles (AgNP) and/or silver ions (Ag⁺) (the silver nanoparticles and/or silver ions may be referred to hereinafter as silver particles), may be in situ encapsulated or trapped in the hydrogel structure forming a silver hydrogel. Alternatively, the silver particles are encapsulated or trapped in a silver hydrogel prior to placement in a biological environment, i.e., ex situ. Simultaneously to the hydrogel formation, the water and/or moisture causes a hydration reaction of the hydratable bioceramic material. The hydrogel acts as a coupling agent that connects the at least one silver nanoparticle and/or at least one silver ion with at least one bioceramic material to form a silver hydrogel bioceramic composite. The release rates of the at least one silver nanoparticle and/or at least one silver ion are engineered by adjusting the hydrogel structure and silver encapsulation processes.

The silver hydrogel bioceramic composite may be formed in situ in a biological environment. The advantage of in situ formation is that the resulting silver hydrogel bioceramic composite is biocompatible, bioactive, antimicrobial, anti-inflammatory, antibacterial, and/or osteoconductive, thereby encouraging bone growth and integration in orthopedic and/or dental applications.

The herein disclosed hydrogel is any three-dimensional, water-swollen network formed from one or more hydrophilic hydrogel formers, which are materials capable of absorbing and retaining water or moisture while maintaining a cohesive, solid-like structure. The herein disclosed hydrogel former may be organic, inorganic, or a combination thereof. An organic hydrogel former is any hydrophilic polymer that undergoes swelling, cross-linking, or polymerization upon contact with water to form a three-dimensional hydrogel network composed primarily of polymeric materials that are biocompatible, meaning they can integrate with or mimic biological tissues. The degree of encapsulation or entrapment depends on the size of the silver particles, the pore size of the hydrogel, and/or the interactions between the hydrogel polymers and the silver particles. The silver ions and/or silver nanoparticles can form ionic or covalent bonds with functional groups in the hydrogel, such as hydroxyl (—OH), carboxyl (—COOH), or amino (—NH₂) groups. Physical interactions, such as hydrogen bonding or van der Waals forces, can also stabilize the silver particles encapsulated or entrapped in the hydrogel.

The organic hydrogel former may be categorized based on its origin (natural or synthetic) and the mechanism of gel formation (physical or chemical crosslinking). The herein disclosed organic hydrogel former may be a natural polymer and/or a synthesized polymer. Natural polymers include, but are not limited to, polysaccharides, alginate, chitosan, cellulose derivatives (hydroxyethyl cellulose or carboxymethyl cellulose are common for hydrogel applications), hyaluronic acid (which are found in connective tissues, and widely used in biomedical and cosmetic applications), proteins, gelatin, collagen, fibrin, starch, agarose, and any combination thereof. Synthetic polymers include, but are not limited to, poly(acrylic acid) (PAA), poly(vinyl alcohol) (PVA), PEG, poly(N-isopropylacrylamide) (PNIPAAm), and any combination thereof.

Inorganic hydrogels are composed of inorganic gel-forming species. The herein disclosed inorganic hydrogel former is any inorganic precursor that forms a hydrated inorganic network through sol-gel condensation or inorganic polymerization. The herein disclosed silver ions and/or silver nanoparticles may be trapped in an inorganic hydrogel, such as a silica hydrogel, calcium silicate hydrogel, aluminum hydrogel, aluminosilicate hydrogel, hydroxyapatite-based hydrogel, and any combination thereof, for controlled release of silver ions and/or silver nanoparticles. The degree of encapsulation or entrapment of silver particles in inorganic hydrogels also depends on the size of the silver particles, the pore size of the hydrogel, and/or the interactions between the inorganic gel-forming species and the silver particles. For example, the inorganic hydrogel former may be: Calcium silicates (C—S-based), including, but not limited to, dicalcium silicate (C₂S), tricalcium silicate (C₃S), calcium metasilicate (CaSiO₃), and combinations thereof; Calcium aluminate hydrogels including 3CaO·Al₂O₃ (C₃A) (tricalcium aluminate, CaO·Al₂O₃(CA), CaO·2Al₂O₃ (CA₂), calcium aluminate cement (CAC), and any combination thereof; Alkali silicate (water glass) hydrogels including sodium silicate (Na₂SiO₃), potassium silicate (K₂SiO₃), lithium silicate (Li₂SiO₃), and any combination thereof; or Metal oxide colloids (sol-gel chemistry) including silica (SiO₂), titania (TiO₂), zirconia (ZrO₂), alumina (Al₂O₃), iron oxides (FeOOH, Fe₂O₃), and any combination thereof. For encapsulating or trapping silver ions and/or silver nanoparticles into an inorganic hydrogel matrix, the materials and synthesis processes are selected to ensure stability, controlled release, and/or uniform distribution of the silver particles. The inorganic hydrogel matrix should be compatible with silver ions and/or silver nanoparticles to avoid unwanted reactions like agglomeration.

The herein disclosed hydrogel may be a hybrid hydrogel. A hybrid hydrogel is a hydrogel whose three-dimensional network is formed from a combination of two or more different organic polymers, two or more different inorganic gel-forming materials, or a combination of organic polymers with inorganic gel-forming materials. For example, the hybrid hydrogel may be formed by combining natural polymers (e.g., alginate) with synthetic ones (e.g., PVA). Hybrid hydrogels may be desirable to enhance mechanical properties and functionality, for example, by selecting or adjusting the ratio of natural polymers to synthetic polymers to achieve a desired degree of crosslinking and porosity for a specific application. The herein disclosed hybrid hydrogel may be a nanocomposite hydrogel, which is a hydrogel incorporating nanoparticles (e.g., silica, carbon nanotubes; typically 1 nm-100 nm in at least one dimension) that interact physically or chemically with the hydrogel matrix. Nanocomposite hydrogels may be desirable to improve mechanical strength or to introduce new functionalities such as conductivity.

The amount of the hydrogel former may be in the range of from about 0.1 wt % to about 20 wt %, from about 0.2 wt % to about 5 wt %, from about 0.5 wt to about 10 wt %, or from about 2 wt % to about 20 wt %, of the total weight of the premixed paste, for example, about 0.1 wt %, about 0.2%, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, or from any one of the recited wt % to any other of the recited wt %. The amount of the hydrogel former in the paste may be limited or reduced to decrease its effect on the physical, chemical, and/or biological properties of bioceramic materials.

The herein disclosed silver hydrogels are hydrogels that encapsulate or entrap the herein disclosed silver nanoparticles and/or silver ions within its pores. A process for encapsulating or trapping silver nanoparticles and/or silver ions in a hydrogel involves a controlled synthesis process to generally evenly distribute the silver particles and retain the silver particles' functional properties, such as antimicrobial activity. This process typically combines hydrogel formation with the synthesis or loading of silver particles. In general, encapsulation or entrapment of silver particles in a hydrogel ex situ includes the following steps of: (1) selecting one or more of the herein disclosed hydrogel formers; (2) preparing a hydrogel precursor solution; (3) incorporating pre-synthesized silver particles into the hydrogel precursor solution; and (4) allowing the hydrogel to form and crosslink so that the silver particles become encapsulated or entrapped in the hydrogel forming a silver hydrogel. The silver hydrogel can subsequently be incorporated with a supplemental hydrogel former into the herein disclosed composition. Hydrogel formation and encapsulation or entrapment of the silver particles can also be done in situ. The silver particles can be directly incorporated into the hydrogel former through uniform mixing, which may prevent localized agglomeration of silver ions and/or silver nanoparticles. Pre-synthesized silver nanoparticles and/or silver ions may alternatively be incorporated after hydrogel formation through a soaking or loading process, wherein the silver particles will diffuse into the hydrogel matrix. Also, in situ formation of silver nanoparticles can be done by loading silver ions into the hydrogel and then introducing one or more reducing agents suitable for reducing silver ions (e.g., sodium borohydride, ascorbic acid) into the hydrogel solution. The reducing agent(s) function by reducing the silver ions into silver nanoparticles within the hydrogel matrix, and then crosslinking the hydrogel will complete the gelation process to lock silver nanoparticles in the matrix.

Controlled release of silver particles from the herein disclosed hydrogel may increase long-term antimicrobial activity while decreasing toxicity. Controlled release of silver particles from the herein disclosed hydrogel will be understood to mean that the silver particles are released at a target rate over a prolonged period, for example, a period of from about 1 month to about 20 years or from about 3 months to about 6 months, for example, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 30 months, about 36 months, about 42 months, about 48 months, about 54 months, about 60 months, about 72 months, about 84 months, about 96 months, about 105 months, about 120 months, about 132 months, about 144 months, about 156 months, about 168 months, about 180 months, about 192 months, about 204 months, about 216 months, about 228 months, about 240 months, or from any one of the recited number of months to any other of the recited number of months. The target rate of release may differ for different types of applications of the premixed paste. The rate of release may be controlled so as to slow and/or accelerate over different stages of its use. In one exemplary embodiment for dental applications, the rate of controlled release of silver particles can be in range of from about 1.0 wt % to about 20 wt % of the total weight of the silver particles released in about the first week, from about 2 wt % to about 50 wt % over about 1 month, from about 3 wt % to about 65 wt % over about 3 months, and from about 5 wt % to about 75 wt % over about 6 months. In another embodiment for medical applications when an increased rate of release is desired, the rate of controlled release of silver particles can be in range of from about 5 wt % to about 50 wt % of the total weight of the silver particles released in about the first week, from about 10 wt % to about 60 wt % over about 1 month, and from about 20 wt % to about 80 wt % over about 3 months.

The release rate of silver particles can be tailored by adjusting the hydrogel composition, crosslinking density, and/or environmental conditions to achieve a target release rate for a given application. For example, the release rate of silver particles encapsulated or trapped in the herein disclosed organic hydrogel is influenced by the types of polymers in the hydrogel, such as natural polymers (e.g., alginate, chitosan) and/or synthetic polymers (e.g., polyacrylamide, polyethylene glycol (PEG)). In general, higher crosslinking creates smaller pores, which in turn reduces the diffusion rate of silver particles and their release rate. Decreasing the size of the silver particles and/or increasing their concentration may increase the silver particle release rate. Silver ions and/or silver nanoparticles can also be released by biodegradable hydrogels as their polymer network breaks down over time. The degradation rate can thus be tailored by modifying the polymer composition or crosslinking. Also, hydrogels can be designed to respond to environmental stimuli, such as pH, temperature, or enzymes. For instance, at low pH (e.g., in infected wounds), the hydrogel network may swell, thereby accelerating silver particle release. Alternatively, enzyme-triggered degradation can lead to localized silver particle release at specific sites. Silver ions bound to functional groups in the hydrogel may be released through ion exchange with other cations (e.g., Na⁺ or H⁺) present in the surrounding environment. The rate of controlled release may be measured by immersing the material in PBS solution and using Atomic Absorption Spectroscopy or Inductively Coupled Plasma Mass Spectrometry to measure the concentration of silver ions and/or silver nanoparticles.

The herein disclosed at least one silver nanoparticle and/or silver ion may be selected from silver salts, silver hydroxide, organic silver compounds, silver complexes with organic ligands, silver alkyl or aryl compounds, silver nanoparticles capped with organic molecules, silver carbene complexes, or any combination thereof. The herein disclosed silver ions may be soluble silver salts such as silver nitrate (AgNO₃), silver acetate (AgC₂H₃O₂), silver fluoride (AgF), silver perchlorate (AgClO₄), silver picolinate (AgC₆H₄NO₂), silver sulfate (Ag₂SO₄), silver citrate (Ag₃C₆H₅O₇), silver lactate (AgC₃H₅O₃), or any combination thereof. The herein disclosed silver nanoparticles may be pre-synthesized or synthesized in situ by reducing silver ions. Stabilizers such as citrate, polyvinylpyrrolidone (PVP), PEG, or any other suitable stabilizer can be incorporated to help prevent aggregation of the silver particles. Other methods known in the art may be used to produce silver nanoparticles.

At least about 10 wt % of the total weight of herein disclosed silver nanoparticles may have a particle size in the range of from about 5 nm to about 500 nm, from about 1 nm to about 200 nm, or from about 2 nm to about 100 nm, for example, about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 125 nm, about 150 nm, about 175 nm, about 200 nm, about 250 nm, about 300 nm, about 350 nm, about 400 nm, about 450 nm, about 500 nm, or from any one of the recited nm to any other of the recited nm.

The amount of the herein disclosed silver nanoparticles and/or silver ions may be in the range of from about 0.01 wt % to about 10 wt %, from about 0.01 wt % to about 5 wt %, from about 0.01 wt % to about 1.0 wt %, or from about 0.1 wt % to about 5.0 wt %, of the total weight of the premixed paste, for example, about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.5 wt %, about 2.0 wt %, about 2.5 wt %, about 3.0 wt %, about 3.5 wt %, about 4.0 wt %, about 4.5 wt %, about 5.0 wt %, or from any one of the recited wt % to any other of the recited wt %.

The herein disclosed hydratable bioceramic material is any biocompatible inorganic powder that reacts with water to form a hardened material. The hydratable bioceramic material may be calcium silicate, dicalcium silicate, tricalcium silicate, calcium aluminates, barium silicates (e.g., tri-barium silicate), strontium silicate, calcium phosphates, calcium sulfate, barium aluminates, strontium aluminates, alkali silicate, alkali aluminate, magnesium silicate, lithium silicate, potassium silicate, ruthenium silicate, or any combination thereof. In some examples, the hydratable bioceramic material is tricalcium silicate (C₃S) and dicalcium silicate (C₂S), which react with water to form calcium silicate hydrates (C—S—H) and calcium hydroxide, providing mechanical strength. In some examples, the hydratable bioceramic material includes calcium aluminates (e.g., CA or C₃A), which hydrate to form compounds such as calcium aluminate hydrates. In some examples, the hydratable bioceramic material includes silica-rich materials (e.g., fly ash, silica fume), which react with lime in the presence of water to form C—S—H. In some examples, the hydratable bioceramic material is calcium sulfate hemihydrate (CaSO₄·0.5H₂O), which reacts with water to form gypsum (CaSO₄·2H₂O).

The amount of the at least one hydratable bioceramic compound may be in the range of from about 5 wt % to about 80 wt %, from about 5 wt % to about 70 wt %, or from about 10 wt % to about 80 wt %, of the total weight of the premixed paste, for example, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, or from any one of the recited wt % to any other of the recited wt %.

The herein disclosed non-aqueous liquid carrier may be any liquid that contains water in an amount sufficiently small that the paste will not undergo hydration and setting when kept in a hermetically-sealed condition. The non-aqueous liquid carrier is used to suspend, dissolve, or disperse ingredients in a formulation without allowing them to hydrate or react prematurely. The non-aqueous liquid carrier is any hydrophilic liquid that facilitates exchange with a water-based fluid, for example, liquid or moisture, in a biological environment by a diffusing process. The non-aqueous liquid carrier may be ethyl alcohol, ethylene glycol, PEG, glycerol liquid, glycerin, liquid organic acids, water-soluble vegetable oil, water-soluble animal oil, fish oil, water-soluble silicon oil, dimethyl sulfoxide, propylene glycol, or any combination thereof. In some examples, the non-aqueous liquid carrier is an alcohol such as 1,3 dihydroxypropane, 1,3-propylene glycol, 1,3-propylenediol, 2-(Hydroxymethyl) ethano, 2-deoxyglycerol, beta-propylene glycol, omega-propanediol, propane-1,3-diol, trimethylene glycol (HOCH2)2ch2, CH2(CH2OH)2, HO(CH2)3oh, HOCH2CH2CH2OH, b-propylene glycol, 1,3-propanediol, or any combination thereof.

For use with the herein disclosed composition, water will preferably be present in the non-aqueous liquid carrier in an amount no greater than about 10 wt % of the total weight of the carrier. For example, the amount of water in the non-aqueous liquid carrier may be in the range of from about 0 wt % to about 10 wt % of the total weight of the carrier, such as 0 wt %, about 1 wt %, about 2 wt %, about 3 wt %, about 4 wt %, about 5 wt %, about 6 wt %, about 7 wt %, about 8 wt %, about 9 wt %, about 10 wt %, or from any one of the recited wt % to any other of the recited wt %. The amount of the non-aqueous liquid carrier may be in the range of from about 5 wt % to about 50 wt % of the total weight of the premixed paste, for example, about 5 wt %, about 10 wt %, about 15 wt %, about 20 wt %, about 25 wt %, about 30 wt %, about 35 wt %, about 40 wt %, about 45 wt %, about 50 wt %, or from any one of the recited wt % to any other of the recited wt %.

The herein disclosed at least one silver nanoparticle and/or at least one silver ion and the herein disclosed hydrogel former may be hydrated to form a core-shell structure, wherein the core comprises the herein disclosed silver particles and the shell comprises the herein disclosed hydrogel. The core-shell structure may be desirable for use in biomedical, dental, pharmaceutical, and/or materials science applications because the core and shell can have distinct or complementary properties. The shell may also comprise one or more suitable bioceramic materials that are hydratable, biocompatible, and/or capable of encapsulating or trapping the silver particles. Such bioceramic materials include, but are not limited to, hydroxyapatite, bioactive glass, calcium silicates, calcium aluminates, and/or zirconia. The herein disclosed core-shell structures may be incorporated into the herein disclosed composition. The herein disclosed core-shell structure may comprise the herein disclosed hybrid hydrogel shell, which may be desirable to leverage the beneficial properties of the inorganic and organic components in a single material. These hybrid hydrogels may exhibit enhanced performance in terms of mechanical strength, chemical functionality, and/or versatility, making them ideal for advanced applications in fields like biomedicine, medical, and/or dental applications. The hybrid hydrogel may thus be used to enhance mechanical strength and/or improve functional properties of the core-shell structure and/or the premixed paste.

The formation of the herein disclosed core-shell structure may involve first the creation of a material with a distinct core material (e.g., silver particles), which is then surrounded by a shell of another material (e.g., an organic hydrogel, an inorganic hydrogel, or a combination thereof). The processes for making the core-shell structure include, but are not limited to, sol-gel process, co-precipitation process, layer by layer assembly, chemical vapor deposition, physical vapor deposition, emulsion or microemulsion techniques, electrochemical deposition, spray coating, self-assembly, or any combination thereof.

Silver nanoparticles have intrinsic optical properties (surface plasmon resonance) that give them a gray to black appearance, depending on size, concentration, and aggregation state. When these silver nanoparticles are encapsulated or trapped or dispersed within a hydrogel network, the polymeric matrix scatters and absorbs light, which reduces direct light reflection from the silver nanoparticles and masks or lightens their apparent color, which helps maintain a whiter or more translucent appearance in the final paste.

The amount of silver particles encapsulated or trapped in the herein disclosed hydrogel core-shell structure may be in the range of from about 0.1% to about 50%, from about 1% to about 30%, or from about 1% to about 20%, of the total weight of the premixed paste, for example, about 0.1 wt %, about 0.5 wt %, about 1 wt %, about 5 wt %, about 10 wt %, about 15 wt %, about 20 wt %, about 25 wt %, about 30 wt %, about 35 wt %, about 40 wt %, about 45 wt %, about 50 wt %, or from any one of the recited wt % to any other of the recited wt %. Silver loaded hydrogel core-shell structure materials with a unique design and structure have significant medical applications and/or dental applications. This design allows the integration of diverse materials, enabling the combination of mechanical strength, biocompatibility, and/or targeted functionality. The applications of the herein disclosed core-shell materials include, but are not limited to, drug delivery systems, tissue engineering scaffolds, wound healing and wound dressing, antimicrobial and antifouling coatings, cancer therapy, antioxidant and/or anti-inflammatory therapies, dental applications, or any combination thereof.

The herein disclosed silver nanoparticles and/or silver ions may be able to kill a wide range of bacteria, fungi, and/or viruses, thereby making silver hydrogels effective in treating and/or preventing infections. The herein disclosed hydrogel may be compatible with biological tissues which make them suitable for various biomedical applications. The herein disclosed hydrogels may control the release rate of silver particles, which can help avoid cytotoxicity while maintaining antimicrobial effectiveness. Whether the released silver particles are cytotoxic will depend on several factors, including but not limited to the concentration of silver particles, the release rate of the silver particles, and the biological tissue type and microenvironment.

Bioactive agents may be co-encapsulated or co-trapped into the herein disclosed silver hydrogel-bioceramic composite, or into the core and/or shell of the herein disclosed core-shell structure, with the silver particles. The bioactive agents may be, but are not limited to, anti-inflammatory drugs, antibiotics, anti-cancer drugs, proteins, DNA, or any combination thereof. The amount of the bioactive agent may be in the range of from about 0.01 wt % to about 10 wt %, or from about 0.05 wt % to about 5 wt %, of the total weight of the premixed paste, for example, about 0.01 wt %, about 0.5 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.5 wt %, about 1.0 wt %, about 1.5 wt %, about 2.0 wt %, about 2.5 wt %, about 3.0 wt %, about 3.5 wt %, about 4.0 wt %, about 4.5 wt %, about 5.0 wt %, or from any one of the recited wt % to any other of the recited wt %.

The herein disclosed compositions may comprise one or more secondary compounds, which can be incorporated into the herein disclosed premixed cement paste, for example when improving the hydrated composition/paste physical properties (such as mechanical strength), chemical stability, and/or biological properties is desirable. The additional secondary compounds may include: tricalcium aluminate (3CaO·Al2O3); tetracalcium aluminoferrite (4CaO·Al2O3·Fe2O3); calcium oxide; ferrite oxide; calcium sulfate dihydrate (CaSO4·2H2O); sodium salts; magnesium salts; strontium salts, ceramics, ceramic fibers, polymers and polymer fibers, metals, metal salts, metal oxides, hydroxide compounds, non-oxide ceramics, biopolymers, or any combination thereof. The amount of the herein disclosed one or more secondary compounds may be less than about 30 wt % of the total weight of the premixed paste, for example, less than about 25 wt %, less than about 20 wt %, less than about 15 wt %, less than about 10 wt %, less than about 5 wt %, or less than about 1 wt %.

The herein disclosed compositions may comprise one or more impurities derived from the herein disclosed components of the compositions. The amount of the one or more impurities may be less than about 10 wt % of the total weight of the premixed paste, for example, less than about 10 wt %, less than about 5 wt %, or less than about 1 wt %. The amount of the one or more impurities may be less than about 30 wt % of the total weight of the at least one hydratable bioceramic material, for example, less than about 25 wt %, less than about 20 wt %, less than about 15 wt %, less than about 10 wt %, less than about 5 wt %, or less than about 1 wt %. Such impurities may include, but are not limited to, iron oxides, magnesia (MgO), potassium oxide, sodium oxide, sulfur oxides, carbon dioxide, water, or any combination thereof.

The herein disclosed compositions may comprise a radiopaque material to improve absorption of X-rays and thus visibility of the implant in X-ray images. The radiopaque material may include, but is not limited to, metals, metal oxides, salts, non-oxides, and any combination thereof. Examples of such radiopaque materials include barium sulfate, zirconium oxide, bismuth oxide, tantalum oxide, tantalum, titanium, stainless steel, alloys, and any combination thereof. The amount of the radiopaque materials may make up less than about 70 wt % of the total weight of the premixed paste, for example, less than about 65 wt %, less than about 60 wt %, less than about 55 wt %, less than about 50 wt %, less than about 45 wt %, less than about 40 wt %, less than about 35 wt %, less than about 30 wt %, less than about 25 wt %, less than about 20 wt %, less than about 15 wt %, less than about 10 wt %, less than about 5 wt %, or less than about 1 wt %.

The herein disclosed compositions may comprise an organic dispersant agent. The organic dispersant agent may be any molecular agent that helps disperse solid particles uniformly in a liquid medium and decrease their clumping, settling, or agglomerating. The organic dispersant agent may be citric acid, sodium citrate, tartaric acid, succinic acid, phosphoric acid, dextrose, mannitol, sucrose, maltose, galactose, lactose, soluble starch, glucose, chitosan, galactose, amylopectin, celluloses, hydroxypropyl methyl cellulose, polyacrylic acids, carbonylmethyl cellulose, biopolymers, organic acids, silane, or any combination thereof. The amount of the organic dispersant agent may be range in the range of from about 0.1 wt % to about 10 wt %, or from about 0.2 wt % to about 5 wt %, of the total weight of the premixed paste, for example, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.5 wt %, about 2.0 wt %, about 2.5 wt %, about 3.0 wt %, about 3.5 wt %, about 4.0 wt %, about 4.5 wt %, about 5.0 wt %, about 5.5 wt %, about 6.0 wt %, about 6.5 wt %, about 7.0 wt %, about 7.5 wt %, about 8.0 wt %, about 8.5 wt %, about 9.0 wt %, about 9.5 wt %, about 10.0 wt %, or from any one of the recited wt % to any other of the recited wt %.

The herein disclosed premixed paste can be prepared by physical mixing processes (nonreactive), chemical processes (reactive), biological processes, and any combination thereof. For example, a premixed paste may be prepared by mixing the solid phases and non-aqueous liquid carrier using a paste mixer. Each of the herein disclosed components of the premixed paste can be combined and mixed together or sequentially. Once prepared, the premixed paste can be loaded into a syringe for administration at the biological site of interest.

The herein disclosed composition may be packaged in a hermetically sealed container. The container helps prevent the composition from contacting water, either in liquid form or, particularly, as airborne vapor, which might otherwise gradually degrade the condition of the cement in the composition. The hermetic packaging enables the premixed paste to remain unused for extended periods without undergoing significant deterioration over time. Accordingly, the packaged paste is fully compatible with modern commercial distribution systems, able to be warehoused and transported by manufacturers, distributors, and end users without requiring special treatment, handling, or other considerations that might otherwise increase the inconvenience and/or cost to an end user.

### EXAMPLES

### Example 1—Premixed Silver/Chitosan/Bioceramic Composite Paste

### Preparation of Silver-Loaded Chitosan Hydrogel:

The materials used for preparing a composite paste included chitosan (biopolymer for hydrogel formation), silver nitrate (AgNO₃) (precursor for silver nanoparticles), sodium hydroxide (NaOH) (for pH adjustment), reducing agent (e.g., ascorbic acid or sodium borohydride for silver nanoparticle synthesis), glycerol or PEG (optional) (as plasticizer for hydrogel flexibility), distilled water, alcohol liquid, calcium silicates, and zirconia.

First, chitosan was dissolved in a dilute acid (e.g., 1% acetic acid) to form a viscous solution (1-2% w/v). The solution was stirred for 4-6 hours at room temperature until fully dissolved, and the pH was adjusted to 5-6 using NaOH to ensure compatibility with silver ions. Second, silver nitrate (AgNO₃) was dissolved in distilled water to prepare a 1 mM solution, to which the silver nitrate solution was added dropwise to the chitosan solution under constant stirring, and a reducing agent (e.g., ascorbic acid or sodium borohydride) was introduced into the mixture to reduce silver ions to silver nanoparticles. Third, the silver nanoparticle-loaded chitosan solution was poured into molds or Petri dishes and gelation was allowed to occur by crosslinking for 24 hours at room temperature. After gelation, the hydrogel was washed with distilled water to remove unreacted silver or acid residues. The samples were dried in an oven at 110° C. for 48 hours, and grinded to a powder. The amount of silver nanoparticles was about 10 wt % of the total weight of the chitosan hydrogel.

The premixed composite paste was prepared by mixing 5 g silver nanoparticle-loaded chitosan hydrogel powder, 50 g calcium silicates (10 wt % dicalcium silicate and 90 wt % tricalcium silicates), 20 g zirconia, and 35 g alcohol.

The premixed composite paste was suitable for dental filling and repairing applications.

The premixed composite paste was biocompatible, bioactive, and very antibacterial.
Setting time in 100% moisture: 6 hours
Compressive strength: 60 MPa
Working time: more than 30 min.

### Example 2

The process for preparing a silver-loaded organic hydrogel/calcium aluminate composite paste for medical and dental applications. This paste combined the antimicrobial properties of silver with the biocompatibility and osteoconductivity of hydroxyapatite, making it suitable for applications such as bone repair, antimicrobial coatings, and dental applications.

The main materials were silver sulfate (Ag2SO4) (i.e., a source of silver ions), gelatin (an organic hydrogel matrix), hydroxyapatite powder (an inorganic bioceramic), and calcium aluminate powder. First, the gelatin was dissolved in warm distilled water (2-5% w/v concentration), and stirred gently at 40-50° C. until fully dissolved to form a hydrogel solution. The silver nanoparticles were prepared by dissolving silver sulfate (1-5 mM) into distilled water, and then added to the hydrogel solution. The hydrogel solution was crosslinked by increasing the temperature to 50° C. The silver nanoparticle-loaded hydrogel was dried at 110° C. for 48 hours, and grinded to a fine powder.

The premixed paste comprised 5 g silver loaded gelation powder, 40 g calcium aluminate, 20 g tantalum oxide, 10 g hydroxyapatite powder, and 25 g b-propylene glycol. The materials were mixed using a paste mixer.

The premixed silicate cement paste was evaluated according to ISO standard 6867:2012:
Setting Time: around 20 min
Working time: >30 min
Radiopacity: Equivalent to 8.5 mm of Al
Flowability: 25 mm
Film thickness: 50 μm
Solubility: <2.0%
Dimension Change: 0.01%
Compressive strength: 60 MPa

Silver nanoparticles/ions release rate was measured by using 3.0 g hard samples and immersing them in 10 ml PBS solution, and refreshing the solution at 1 week, 4 weeks, 3 months, and 6 months. Silver nanoparticles in the solution were measured using Inductively Coupled Plasma Optical Emission Spectroscopy (ICP-OES).

| Time | 1 week | 4 week | 3 month | 6 month |
|---|---|---|---|---|
| Cumulative | 2.2 | 4.8 | 9.3 | 16.5 |
| AgNP Release | | | | |
| (%) | | | | |

The paste was effective at killing the bacteria.

### Example 3

The composite paste was prepared by mixing 0.4 g silver nanoparticles (average D=20 nm), 10 g hydroxypropyl methyl cellulose (as the hydrogel former to encapsulate the silver ions), 10 g calcium phosphate, 50 g tricalcium silicate, 30 g Bismuth oxide, 35 g PEG (molecular weight 400, Sigma-Aldrich). Materials were mixed by using a paste mixer.

When the composite paste was injected into a root canal, the water and/or moisture from the biological environment diffused into the composite paste and the hydroxypropyl methyl cellulose hydrogel encapsulated silver ions to allow for controlled release of the silver ions.

The premixed silicate cement paste was evaluated according to ISO standard 6867:2012:
Setting Time: around 3 hrs.
Working time: >30 min.
Radiopacity: Equivalent to 9.0 mm of Al
Flowability: 25 mm
Film thickness: 50 μm
Solubility: <2.0%
Dimension Change: 0.01%
Compressive strength: 58 MPa

### Example 4

The silver nanoparticles were encapsulated in an aluminum hydrogel structure for controlled release. The aluminum hydrogel was prepared by dissolving 10 g Boehmite (AlOOH) in 100 g distilled water and then dissolving 1 g ANO3 powder into Boehmite solution. The crosslinking of the AlOOH sol solution was achieved by adjusting the pH to around 10. The AlOOH/Ag gel was dried at 110° C. for 72 hours, and then grinded to a fine powder.

The premixed paste comprised 5 g silver-loaded AlOOH gel powder, 30 g dicalcium silicate, 30 g Zirconia, 10 g dicalcium phosphate, and 30 g PEG. The materials were mixed using a paste mixer.

The AlOOH gel allowed for controlled release of silver nanoparticles and/or silver ions to kill the bacteria.

### Example 5

Premixed paste of silver silica hydrogel, calcium phosphate, calcium silicate, zirconia, and b-propylene glycol.

Silver-loaded silica hydrogel was prepared by mixing tetraethyl orthosilicate (TEOS), ethanol, and deionized water in a molar ratio of approximately 1:4:2 in a beaker, and the solution was stirred vigorously for about 1 hour at room temperature to allow hydrolysis and condensation reactions to form a silica sol.

Silver nitrate (AgNO₃) was dissolved in deionized water to prepare a silver ion solution (e.g., 0.01-0.1 M). The silver nitrate solution was then slowly added to the silica sol while stirring. The silver ions were incorporated into the silica network. The mixture sat at room temperature or under slightly elevated temperatures (e.g., 40-50° C.) until a gel was formed. The gel was aged for 12-24 hours to strengthen the silica network. The gel was dried under ambient conditions or in an oven at low temperatures (e.g., 60° C.), and then grinded to a fine powder.

The premixed paste was prepared by mixing 5 g silver silica gel powder, 20 g calcium phosphate, 25 g calcium silicate (100% tricalcium silicate), 25 g zirconia, and 28 g b-propylene glycol. The paste was mixed using a paste mixer for 30 min. The premixed cement paste was evaluated according to ISO standard 6867:2012:
Setting Time: around 2 hrs.
Working time: >30 min.
Radiopacity: Equivalent to 7.0 mm of Al
Flowability: 22 mm
Film thickness: 50 μm
Solubility: <2.0%

### Example 6

### Silver Nanoparticles (AgNP) Core/Alginate Hydrogel Shell/Calcium Silicate Cement.

Core materials comprising 100 mg stable silver nanoparticles were prepared using an aqueous dispersion of AgNPs that was already stabilized (e.g., citrate-coated). The shell structure was prepared using 1.0 g alginate which is a naturally-derived polymer that stayed liquid in its sodium salt form. When the AgNPs were mixed into this alginate solution, the nanoparticles became uniformly suspended within the precursor of the future shell.

Alginate forms a hydrogel when it encounters multivalent cations (e.g., Ca²⁺). Accordingly, the alginate chains crosslinked when exposed to Ca²⁺ which produced a 3D hydrogel shell that trapped the AgNPs inside. The shell formed where the alginate met the Ca²⁺. The end result was that each AgNP (or AgNP cluster) became surrounded by a shell composed of an ionically-crosslinked alginate network.

The paste was prepared by mixing 40 g tricalcium silicate, 20 g zirconia, 1.1 g AgNP core/alginate shell material, 10 g tri-barium silicate, and 28.9 g PEG, which were then mixed using a paste mixer.
Physical properties:
Setting Time: around 4 hrs.
Working time: >30 min.
Radiopacity: Equivalent to 8.0 mm of Al
Flowability: 27 mm
Film thickness: 20 μm
Solubility: <2.0%

The silver nanoparticles release rate was measured by immersing 1.0 g hard samples in 10 ml PBS solution, and refreshing the PBS solution at 1 week, 4 weeks, 3 months, and 6 months. Silver nanoparticles (AgNPs) in the solution were measured using surface plasmon resonance (SPR), typically at about 400-450 nm.

| Time | 1 week | 4 week | 3 month | 6 month |
|---|---|---|---|---|
| Cumulative | 1.2 | 3.1 | 6.0 | 10.6 |
| AgNP Release | | | | |
| (%) | | | | |

### APPENDIX

Present invention discloses a composition of silvery hydrogel bioceramic composite, manufacture process, and the applications. A silver hydrogel bioceramic composite is an advanced material that combines the properties of silver nanoparticles, hydrogels, and bioceramic components to create a multifunctional system with applications in biomedicine, tissue engineering, dental applications, and wound care. In present invention, a silver hydrogel is made by incorporating silver nanoparticles (AgNPs) or silver ions into its hydrogel structure. In fact, the hydrogel structure is a coupling agent to connect silver nanoparticle or ions with ceramic materials to form silver hydrogel bioceramic composite. The release rate of nanosilver and/or nanosilver ions are engineered by just hydrogel structure and silver encapsulation process.

In another embedment, the silver hydrogel bioceramic composition can be in-situ formation in biological environment. The advantage of in situ formation of silvery hydrogel bioceramic composite are biocompatible, bioactive, antimicrobial, anti-inflammatory, and antibacterial properties to promote osteoconductive, encouraging bone growth and integration in orthopedic or dental applications.

Silver organic hydrogels are composite materials that combine hydrogels—a three-dimensional, water-absorbent polymer network—with silver, typically in the form of silver nanoparticles (AgNPs) or silver ions (Ag⁺). In one embodiment, silver ions (Ag⁺) are incorporated into the hydrogel matrix, and reducing agents (e.g., sodium borohydride, ascorbic acid) are used to convert them into silver nanoparticles within the hydrogel structure. Also, pre-synthesized silver nanoparticles are encapsulated into the hydrogel matrix during its formation or are embedded afterward through soaking or loading processes. Hydrogels are composed of polymer chains that create a porous network. This network can trap and immobilize silver nanoparticles or ions within its pores. The degree of encapsulation depends on the size of the silver particles, the pore size of the hydrogel, and the interactions between the hydrogel polymers and the silver. In other embodiment, silver ions or nanoparticles can form ionic or covalent bonds with functional groups in the hydrogel, such as hydroxyl (—OH), carboxyl (—COOH), or amino (—NH₂) groups. Physical interactions, such as hydrogen bonding or van der Waals forces, can also stabilize the encapsulation.

Encapsulations of silver in hydrogel structure is related to polymer types, such as natural polymers (e.g., alginate, chitosan) or synthetic polymers (e.g., polyacrylamide, PEG) influence the interaction with silver and the release rate. In general, the higher crosslinking creates smaller pores, reducing the diffusion rate of silver. Smaller nanoparticles and higher concentrations may lead to faster release.

In other embodiment, silver ions or silver nanoparticles are capsulated into inorganic hydrogel, and controlled release of silver lons or nano silver particulars, such silica hydrogel, calcium silicate hydrogel, aluminum hydrogel, etc, and mixture thereof. For encapsulating silver ions or silver nanoparticles (AgNPs) into an inorganic hydrogel matrix, the materials and synthesis processes are selected to ensure stability, controlled release, and uniform distribution of the silver. The matrix of inorganic hydrogel is selected from group of silica-based hydrogels, aluminosilicates, or hydroxyapatite-based hydrogels. Also, the hydrogel matrix should be compatible with silver ions or nanoparticles to avoid unwanted reactions like agglomeration. The silver ions are selected from soluble silver salts such as silver nitrate (AgNO₃) or silver acetate, Silver Fluoride (AgF), Silver Perchlorate (AgClO₄), Silver Sulfate (Ag₂SO₄), and mixture thereof. The silver nano particles are synthesized or procure well-dispersed nanoparticles in a suitable medium. Stabilizers like citrate, PVP, or PEG can help prevent aggregation.

In other embodiment, it is very important to make the silver ion solution and/or nanosilver suspension stable and homogenesis in inorganic gelation process. The uniform mixing process is to prevent localized agglomeration of silver ions or particles. The crosslink strong hydrogel network trap silver ions/nanoparticles Also, the inorganic hydrogel can be synthesized first and immerse it in a silver ion or nanoparticle solution. The silver will diffuse into the hydrogel matrix.

The controlled release of silver from hydrogels/bioceramic composite structure is crucial for ensuring long-term antimicrobial activity while minimizing toxicity. The release mechanism is influenced by the hydrogel composition, crosslinking density, and environmental conditions. The silver ions or nanoparticles in hydrogel/bioceramic composite diffuse out of the hydrogel matrix through its pores. The rate depends on the hydrogel's pore size and the diffusivity of the silver species. Also, silver ions or sil can be released by biodegradable hydrogels as their polymer network breaks down over time. The degradation rate can be tailored by modifying the polymer composition or crosslinking. Also, hydrogels can be designed to respond to environmental stimuli, such as pH, temperature, or enzymes. For instance: At low pH (e.g., in infected wounds), the hydrogel network may swell, accelerating silver release. Enzyme-triggered degradation can lead to localized silver release at specific sites. Silver ions bound to functional groups in the hydrogel may be released through ion exchange with other cations (e.g., Na⁺ or H⁺) present in the surrounding environment.

The premixed silver hydrogel material paste in present invention include as least one hydrogel materials, at least one nano-silver and/or silver ions, at least one hydratable bioceramic materials, and/or at least non-aqueous liquid carrier. When the premixed paste said in present invention are placed in biological environment, the non-aqueous liquid carrier exchange with water and/or moisture from biological environment, the hydrogel materials absorb the water to produce the hydrogel, the nano-silver and/or silver ions are in-situ incorporated into hydrogel structure, hydration reaction of hydratable bioceramic materials take place simultaneously. The hydrogel structure in present invention is a coupling agent to contact nano silver and/or silver ions.

The hydrogel formers in present invention include, but not limited, Alginate, chitosan, gelatin, or hyaluronic acid, Polyvinyl alcohol (PVA), polyacrylamide, polyethylene glycol (PEG), citric acid, sodium citrate, tartaric acid, succinic acid, phosphoric acid, dextrose, mannitol, sucrose, maltose, galactose, lactose, soluble starch, glucose, chitosan, galactose, amylopectin, celluloses, hydroxypropyl methyl cellulose, polyacrylic acids, carbonylmethyl cellulose, biopolymers, organic acids, silane, and mixtures of thereof. The hydratable bioceramic materials in present invention include, but not limited, Calcium silicate, dicalcium silicate, tricalcium silicate, calcium aluminates, barium silicates, strontium silicate, calcium phosphate cement, calcium sulfate, and mixture thereof.

Tricalcium silicate (C₃S) and dicalcium silicate (C₂S) react with water forms calcium silicate hydrates (C—S—H) and calcium hydroxide, providing mechanical strength. The calcium aluminates (e.g., CA or C₃A) that hydrate to form compounds like calcium aluminate hydrates. Silica-rich materials (e.g., fly ash, silica fume) that react with lime in the presence of water to form C—S—H. Calcium Sulfate Hemihydrate (CaSO₄·0.5H₂O) reacts with water to form gypsum (CaSO₄·2H₂O).

According to an embodiment, the hydratable bioceramic compounds are in the range of 5 wt %-80 wt % of the premixed paste, or 5 wt %-70 wt %, or 10 wt %-80 wt %, while the non-aqueous liquid of the present invention is the range of 5 wt %-45 wt % of premixed paste. According to an embodiment, the non-aqueous liquid carrier is a hydrophilic liquid, to facilitate the exchange with water in the biological environment, by a diffusing process.

The non-aqueous liquid carriers in present invention include, but not limited to, ethyl alcohol, ethylene glycol, polyethylene glycol (PEG), glycerol liquid, glycerin, liquid organic acids, water-soluble vegetable oil, water-soluble animal oil, fish oil, water-soluble silicon oil, dimethyl sulfoxide, propylene glycol, etc and mixture thereof. In another embodiment, alcohols in present invention include, 1,3 Dihydroxypropane, 1,3-Propylene glycol, 1,3-Propylenediol, 2-(Hydroxymethyl) ethano, 2-Deoxyglycerol, beta-Propylene glycol, omega-propanediol, Propane-1,3-diol, Trimethylene glycol (HOCH2) 2ch2, 1,3-PROPANDIOL, CH2(CH2OH)2, HO(CH2)3oh, HOCH2CH2CH₂OH, b-Propylene glycol, B-propylene glycol, 1,3-Propanediol, and mixture thereof.

In one embodiment, the hydrogel materials in composition of premixed paste are used as a coupling agent to connect the nano-silver and/or silver ions. The hydrogel materials in composition will not act as structural component. The amount of hydrogel materials in the composition should be limited to not affect the physical, chemical, and biological properties of bioceramic materials. Hydrogel materials in the composition is in the range of 0.1 wt %-20 wt %, 0.2 wt %-5 wt %, 0.5 wt-10 wt %, 2 wt %-20 wt %, and mixture thereof.

One embedment, a core-shell structure composite material was designed to have at least one silver core and at least one hydrogel shell. The silver core/hydrogel shell structures said in present invention can be used in biomedical, dental, pharmaceutical, and materials science applications because the core and shell can have distinct or complementary properties. The shell in present invention are bioceramic composition that are biocompatible, meaning they can integrate with or mimic biological tissues, including hydroxyapatite, bioactive glass, calcium silicates, calcium aluminates and zirconia etc. The silver loaded hydrogel cores include, but not limited to, organic hydrogel, inorganic hydrogel, and mixture thereof. The silver was encapsulated into hydrogel structure for controlled released and mask the color of siliver nanoparticles.

One embodiment, organic hydrogels said in present invention are primarily composed of polymeric materials that are biocompatible and can form a three-dimensional network. These materials are typically categorized based on their origin (natural or synthetic) and the mechanism of gel formation (physical or chemical crosslinking). These materials include natural polymer and synthesized polymers, such as Polysaccharides, Alginate, Chitosan, Cellulose Derivatives (Hydroxyethyl cellulose or carboxymethyl cellulose are common for hydrogel applications), Hyaluronic Acid (Found in connective tissues; widely used in biomedical and cosmetic applications), Proteins, Gelatin, Collagen, Fibrin, Starch, Agarose, and mixtures thereof. Synthetic Polymers includes, but not limited to, Poly(acrylic acid) (PAA), Poly(vinyl alcohol) (PVA), Poly(ethylene glycol) (PEG), Poly(N-isopropylacrylamide) (PNIPAAm), and mixtures thereof. The hybrid hydrogels are used as hydrogel shell in present invention, such as Natural-Synthetic Composites (Combining natural polymers (e.g., alginate) with synthetic ones (e.g., PVA) to enhance mechanical properties and functionality) and Nanocomposite Hydrogels (Incorporation of nanoparticles (e.g., silica, carbon nanotubes) to improve mechanical strength or introduce new functionalities like conductivity).

In another embodiment, the inorganic hydrogel shell materials said in present invention can used as bioceramic shell materials as well. Also, hybrid hydrogel shell said in present invention is to combine both inorganic and organic components in a single material to leverage the best properties of each. These hydrogels exhibit enhanced performance in terms of mechanical strength, chemical functionality, and versatility, making them ideal for advanced applications in fields like biomedicine, medical, and dental applications. The advantages of hybrid hydrogel are to enhance mechanical strength, improve functional properties etc.

The process for encapsulating silver nanoparticles (AgNPs) or silver ions into a hydrogel involves a controlled synthesis process to ensure the silver is evenly distributed and retains its functional properties, such as antimicrobial activity. The process typically combines hydrogel formation with the synthesis or loading of silver nanoparticles. In general, encapsulation of silver in hydrogel includes following steps, 1, to select hydrogel materials as hydrogel former, such as organic hydrogel, inorganic hydrogel, and the mixtures thereof. 2. To dissolve hydrogel to hydrogel precursor, 3, to incorporate silver into hydrogel precursor. 4, to embed in hydrogel and crosslink the hydrogel. The silver nanoparticles can be directly incorporated into the hydrogel precursors. Also, in-situ formation of silver nanoparticle is to load silver ions into the hydrogel, and then to introduce reducing agents into the hydrogel solution. The silver ions will be reduced to nanoparticles within the hydrogel matrix, and then crosslink the hydrogel to complete the gelation process to lock silver nanoparticles in the matrix.

In another embodiment, since silver nanoparticles are gray or black in color, encapsulating them within a hydrogel matrix can partially or completely mask their color, thereby reducing the visible grayish appearance of the materials.

Silver nanoparticles (AgNPs) have intrinsic optical properties (surface plasmon resonance) that give them a gray to black appearance, depending on size, concentration, and aggregation state. When these nanoparticles are encapsulated or dispersed within a hydrogel network, the polymeric matrix scatters and absorbs light, which to reduce direct light reflection from the nanoparticles, to mask or lightens their apparent color, and to helps maintain a whiter or more translucent appearance in the final paste.

One embodiment, the bioactive agents can co-capsulated into hydrogel shell structure with silver together. The bioactive agents said in present invention include, but not limited to, anti-inflammatory drugs, antibiotics, anti-cancer drugs, proteins, DNA and mixture thereof.

The weight percentage of silver encapsulated in hydrogel core/shell composition said in present invention is in the range of 0.1% to 50%, preferable range 1%-30%, another preferable range is 1%-20%.

The formation of core-shell structured materials said in present invention typically involved the creation of a material with a distinct core surrounded by a shell of another material. The processes for making core-shell materials include, but not limited to, Sol-gel process, co-precipitation process, layer by layer assembly, chemical vapor deposition, physical vapor deposition, emulsion or microemulsion techniques, electrochemical deposition, spray coating, self-assembly, and mixture thereof.

In one embodiment, the silver loaded hydrogel core/shell structure materials with unique design and structure have significant medical applications and dental applications. This design allows the integration of diverse materials, enabling the combination of mechanical strength, biocompatibility, and targeted functionality. The applications of core-shell materials in present invention include, but not limited to, drug deliver system, tissue engineering scaffolds, wound healing and wound dressing, antimicrobial and antifouling coatings, cancer therapy, antioxidant and anti-inflammatory therapies, dental applications, and mixtures thereof.

One embodiment, the nano-silver and or silver ions are able to kill a wide range of bacteria, fungi, and viruses, making silver hydrogels effective in preventing infections. Hydrogels compatibility with biological tissues, making them suitable for various biomedical applications. The hydrogel materials can control the release rate of silver ions, which can help avoid cytotoxicity while maintaining antimicrobial effectiveness.

In present invention, at least 10 wt % of particle size of nano-silver is in the range of 5 nm (nanometer)-500 nm (nanometer), 1 nm-200 nm, and 2 nm-100 nm. The silver ions are selected from group silver salt, silver hydroxide, and organic silver compounds. Silver salts in present invention include, but not limited to AgNO3 AgF AgClO4, Ag2SO4, and AgC2H3O2. The silver organic compound in present invention include, but not limited to Silver Acetate, Silver Citrate, Silver Lactate, Silver Nanoparticles Capped with Organic Molecules, Silver Carbene Complexes, Silver Picolinate, Silver Alkyl or Aryl Compounds, Silver Complexes with Organic Ligands and mixture of thereof.

The nano-silver and or silver ions in composition is the range of 0.01 wt %-10 wt %, one preferable range of 0.01 wt %-5 wt %, 0.01 wt %-1.0 wt %, another preferable range of 0.1-5.0 wt %.

According to an embodiment, one or more additional secondary compounds are incorporated into the premixed cement paste. The additional secondary compounds may include tricalcium aluminate (3CaO·Al2O3); tetracalcium aluminoferrite (4CaO·Al2O3·Fe₂O₃); calcium oxide; ferrite oxide; calcium sulfate dihydrate (CaSO4·2H2O); sodium salts; magnesium salts; and/or strontium salts, and comprise less than 30 wt % by weight of the cement in the premixed paste composition. The premixed cement paste may also contain a number of impurities from the original raw materials, preferably in an amount less than 10 wt % of the paste in the cement composition or 30 wt % of the cement powder in the cement composition. Such impurities may include, for example, iron oxides, magnesia (MgO), potassium oxide, sodium oxide, sulfur oxides, carbon dioxide, water, etc.

One embodiment, radiopaque materials may be added to the paste composition to improve absorption of X-rays and thus visibility of the implant in X-ray images. The radiopaque materials that may be used include, for example, metals, metal oxides, salts, non-oxides, and mixtures thereof. Examples of such additive materials include barium sulfate, zirconium oxide, bismuth oxide, tantalum oxide, tantalum, titanium, stainless steel, alloys, and mixtures thereof, which, according to an embodiment, make up less than about 70% of the cement powder composition.

As noted above, the injectable premixed cement paste of the present invention does not set and harden in a hermetically sealed package because the reactions between hydratable cement compound and water only take place when exposed to an aqueous environment. After the cement paste is placed in contact with a physiological solution, a diffusional exchange of the non-aqueous carrier with the aqueous physiological solution, thereby exposing the premixed paste water and initiating the chemical reaction that transitions the cement to a hydrogel.

According to an embodiment, the premixed paste is packaged in a hermetically sealed container. The container prevent prevents the paste from contacting water, either in liquid form or, particularly, as airborne vapor, which might otherwise gradually degrade the condition of the cement in the paste. The hermetic packaging enables the premixed paste to remain unused for extended periods without undergoing significant deterioration over time. Accordingly, the packaged paste is fully compatible with modern commercial distribution systems, able to be warehoused and transported by manufacturers, distributors, and end users without requiring special treatment, handling, or other considerations that might otherwise increase the inconvenience and/or cost to an end us

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1. A premixed paste for use in medical and/or dental applications, comprising:
at least one silver nanoparticle and/or at least one silver ion;
at least one hydrogel former;
at least one hydratable bioceramic material; and
at least one non-aqueous liquid carrier that is hydrophilic and able to undergo an exchange with a water-based liquid when the premixed paste is exposed to an environment where water-based liquids are present.

2. The premixed paste for use of claim 1, wherein the at least one silver nanoparticle and/or at least one silver ion is encapsulated or entrapped in a hydrogel.

3. (canceled)

4. The premixed paste for use of claim 1, wherein the at least one hydrogel former comprises an organic hydrogel former, an inorganic hydrogel former, or any combination thereof.

5. The premixed paste for use of claim 1, wherein the at least one hydrogel former forms a core/shell structure.

6. (canceled)

7. The premixed paste for use of claim 1, wherein the at least one silver nanoparticle and/or silver ion is a silver salt, a silver hydroxide, an organic silver compound, a silver carbene complex, a silver nanoparticle capped with one or more organic molecules, or any combination thereof.

8. The premixed paste for use of claim 7, wherein the silver salt is silver nitrate (AgNO₃), silver acetate (AgC₂H₃O₂), silver fluoride (AgF), silver perchlorate (AgClO₄), silver sulfate (Ag₂SO₄), silver citrate (Ag₃C₆H₅O₇), silver lactate (AgC₃H₅O₃), silver picolinate (AgC₆H₄NO₂), or any combination thereof.

9. The premixed paste for use of claim 7, wherein the organic silver compound is a silver alkyl and/or aryl compound.

10. The premixed paste for use of claim 1, wherein the premixed paste further comprises at least one reducing agent.

11. (canceled)

12. The premixed paste for use of claim 1, wherein the premixed paste further comprises at least one stabilizer.

13. (canceled)

14. The premixed paste for use of claim 1, wherein the at least one silver nanoparticle and/or silver ion is present in an amount of at least 0.01 wt % of the total weight of the premixed paste.

15. The premixed paste for use of claim 1, wherein the at least one silver nanoparticle and/or silver ion is present in an amount of at least 0.1 wt % of the total weight of the premixed paste.

16. The premixed paste for use of claim 1, wherein the at least one silver nanoparticle has a particle size in the range of from about 5 nm to about 500 nm.

17. The premixed paste for use of claim 1, wherein the at least one hydratable bioceramic material is calcium silicate, dicalcium silicate, tricalcium silicate, calcium aluminate, barium aluminate, barium silicate, calcium phosphate, strontium silicate, strontium aluminate, alkali silicate, alkali aluminate, magnesium silicate, lithium silicate, sodium silicate, potassium silicate, ruthenium silicate, or any combination thereof.

18. The premixed paste for use of claim 1, wherein the at least one hydratable bioceramic material is present in an amount of less than about 80 wt % of the total weight of the premixed paste.

19. The premixed paste for use of claim 1, wherein the non-aqueous liquid carrier contains water in an amount no greater than about 10 wt % of the total weight of the carrier.

20. The premixed paste for use of claim 1, wherein the non-aqueous liquid carrier is present in an amount of no more than 50 wt % of the total weight of the premixed paste.

21. The premixed paste for use of claim 1, wherein the non-aqueous liquid carrier is ethyl alcohol, ethylene glycol, polyethylene glycol, glycerol liquid, glycerin, liquid organic acids, water-soluble vegetable oil, water-soluble animal oil, fish oil, water-soluble silicon oil, dimethyl sulfoxide, propylene glycol, 1,3-dihydroxypropane, 1,3-propylene glycol, 1,3-propylenediol, 2-(hydroxymethyl) ethano, 2-deoxyglycerol, beta-propylene glycol, omega-propanediol, propane-1,3-diol, trimethylene glycol (HOCH2)2ch2, CH2(CH2OH)2, HO(CH2)3oh, HOCH2CH2CH2OH, b-propylene glycol, 1,3-propanediol, or any combination thereof.

22. The premixed paste for use of claim 1, wherein the premixed paste further comprises one or more secondary compounds, wherein the one or more secondary compounds are ceramics, ceramic fibers, polymers and polymer fibers, metals, metal salts, metal oxides, hydroxide compounds, non-oxide ceramics, biopolymers, or any combination thereof.

23. (canceled)

24. (canceled)

25. The premixed paste for use of claim 1, wherein the premixed paste further comprises one or more organic dispersant agents.

26. (canceled)

27. The premixed paste for use of claim 1, wherein the premixed paste further comprises one or more bioactive agents.

28. (canceled)

29. The premixed paste for use of claim 1, wherein the rate of release of the at least one silver nanoparticle and/or silver ion is in the range of from about 1.0 wt % to about 20 wt % over about 1 week, from about 2 wt % to about 50 wt % over about 1 month, from about 3 wt % to about 65 wt % over about 3 months, from about 5 wt % to about 75 wt % over about 6 months, or any combination thereof, of the total weight of the premixed paste.

30. A method of forming a premixed paste for use in medical and/or dental applications, the method comprising the steps of:
mixing:
at least one silver nanoparticle and/or at least one silver ion;
at least one hydrogel former;
at least one hydratable bioceramic material; and
at least one non-aqueous liquid carrier that is hydrophilic and able to undergo an exchange with a water-based fluid when the premixed paste is exposed to an environment where water-based fluid is present.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. A method for forming a cementitious mass in medical and/or dental applications, said method comprising the steps of:
providing a premixed paste, said premixed paste comprising:
at least one silver nanoparticle and/or at least one silver ion;
at least one hydrogel former;
at least one hydratable bioceramic material; and
at least one non-aqueous liquid carrier that is hydrophilic and able to undergo an exchange with a water-based liquid when the premixed paste is exposed to an environment where water-based fluid is present, and
placing said premixed paste in a physiological environment so that said at least one non-aqueous liquid carrier undergoes exchange with an aqueous physiological fluid so that said paste hydrates in said physiological environment.

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)
